(19) United States
(12) Patent Application Publication
(10) Pub. No.: US 2024/0383271 A1
(43) Pub. Date: Nov. 21, 2024

(54) SINGLE-USE WAX MARKING TOOL

(71) Applicant: SERA Solutions, LLC, Flagstaff, AZ (US)

(72) Inventor: Melissa-Ann Ares, Flagstaff, AZ (US)

(73) Assignee: SERA Solutions, LLC, Flagstaff, AZ (US)

(21) Appl. No.: 18/631,469

(22) Filed: Apr. 10, 2024

Related U.S. Application Data
(60) Provisional application No. 63/503,470, filed on May 20, 2023.

(51) Int. Cl. B43K 23/016; A61B 90/00; B43K 19/00

(52) U.S. Cl. CPC B43K 23/016; A61B 90/39; B43K 19/00; A61B 2090/395

(57) ABSTRACT

A single-use wax writing tool with a colored wax tip that is used once and disposed. In accordance with the present disclosure a single-use wax marking tool comprises a body having a tapered tip filled with a colored wax. The tapered tip may be an integrated part of the body or a separate component that is attached to the body to form the single-use wax marking tool. The body may be hollow or solid. The tapered tip comprises a hollow portion to receive the wax. The tapered tip may have structures that help secure the wax within the tapered tip. The wax may generally be any desirable color and may be biodegradable. In some configurations, the body further comprises a label, which may be a printed label or may be an embossed label or a debossed label, all of which may also be biodegradable.

### CROSS REFERENCE TO RELATED APPLICATIONS

This patent application claims priority under 35 U.S.C. § 119(e) to U.S. Provisional Patent Application No. 63/503,470, entitled “Single-Use Wax Marking Tool,” filed May 20, 2023, which is incorporated by reference in its entirety for all purposes.

### TECHNICAL FIELD

The present disclosure relates generally to a writing tool with a wax tip that can be used once and then disposed.

### BACKGROUND

Various health care offices, including doctors' offices, emergency rooms, and laboratories such as neurodiagnostic labs typically see a variety of patients of varying degrees of health and perform a variety of tests on the patients. For example, such tests include electroencephalograms (EEGs), a diagnostic test used to evaluate the electrical activity of the brain. The EEG is performed by first drawing an accurate map on a patient's scalp to identify electrode placement. This map is typically marked with a multiuse wax pencil (also known as “China markers”) due to their water repellant nature, yet demonstrating generally easy removal. This is especially important when treating ill patients who may be sweating profusely.

As illustrated in FIG. 1, these prior art wax pencils are generally made of a perforated paper wrap, a string pull tab, and a wax core. This type of pencil is “sharpened” by peeling the paper wrap away and exposing more wax. The porous nature of these pencils creates countless crevices in which infectious microbes can remain in, even when the exterior is wiped with bleach. One such wax pencil can easily be used on hundreds of patients including patients with HIV, Hepatitis C, Scabies and the like. The standard disinfecting protocol being to wipe the wax pencil with bleach and peel down an upper layer of paper.

Additionally, the standard wax pencil is also not labeled for safe use on skin or hair, nor does it have the required Instructions for Use (IFU) to be considered an approved multiuse tool within accredited hospitals. When being used on patients with delicate skin, considerable pressure needs to be applied as the standard wax pencils formula does not mark well on skin. This can contribute to skin breakdown, and an increased infection risk.

These pencils are often reused on outpatients and inpatients, including both healthy and immunocompromised patients. While an EEG is generally considered a non-invasive test, they are regularly conducted on trauma patients leaving pencils covered in blood and skin cells and there is no guarantee that wiping with bleach and peeling down the upper layer of paper has eradicated any blood borne pathogens within the porous, multi-layered paper of existing wax pencils currently being used. Moreover, existing wax pencils often have unknown ingredients and the safety of use on skin is not specified. In light of these concerns, some health care facilities have gone so far as to ban the use of the same wax pencils between patients.

Accordingly, a single-use wax marking tool which helps eliminate contamination such as nosocomial and other infections acquired from multi-use wax pencils is desirable.

### SUMMARY

The present disclosure provides a single-use wax writing tool with a colored wax tip that is used once and then disposed of, creating less waste than with prior art wax pencils, while reducing issues related to cross-contamination. For example, in accordance with the present disclosure a single-use wax marking tool comprises a body having a tapered tip (or end) filled with a colored wax. The tapered tip may be an integrated part of the body or a separate component that is attached to the body to form the single-use wax marking tool. The body may be hollow or may be solid, though in embodiments with a solid body the tapered tip comprises a hollow portion to receive the wax. In various embodiments, the tapered tip may have structures that help secure the wax within the tapered tip, though the wax may also be press fit into the tapered tip. The wax may generally be any desirable color (including the color black) and may be biodegradable. In some configurations, the body further comprises a label, which may be a printed label or may be an embossed label or a debossed label, all of which may also be biodegradable.

### BRIEF DESCRIPTION OF THE DRAWINGS

The accompanying drawings are included to provide a further understanding of the disclosure and are incorporated in and constitute a part of this specification, illustrate embodiments of the disclosure, and together with the description serve to explain the principles of the disclosure, wherein:

FIG. 1 is a prior art multi-use wax pencil.

FIG. 2 is a front view of a single-use wax pencil having an integral, one-piece body and hollow tapered tip in accordance with the present disclosure.

FIG. 3 is a cross-sectional view of another embodiment of a single-use wax pencil having a separate, attachable and detachable hollow tapered tip in accordance with the present disclosure.

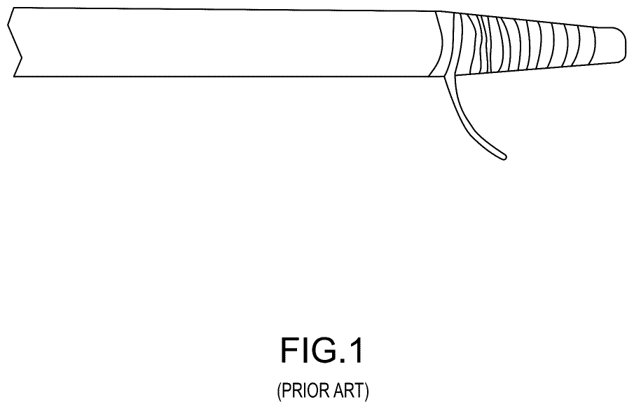

### DETAILED DESCRIPTION OF THE ILLUSTRATED EMBODIMENTS

Persons skilled in the art will readily appreciate that various aspects of the present disclosure can be realized by any number of methods and systems configured to perform the intended functions. Stated differently, other methods and systems can be incorporated herein to perform the intended functions. It should also be noted that the accompanying drawing figures referred to herein are not all drawn to scale but may be exaggerated to illustrate various aspects of the present disclosure, and in that regard, the drawing figures should not be construed as limiting. Finally, although the present disclosure can be described in connection with various principles and beliefs, the present disclosure should not be bound by theory.

In general, the present disclosure provides devices, systems and methods for writing tool with a replaceable colored wax tip that can be used once and disposed of with less waste associated than a typical wax pencil. For example, writing tools in accordance with the present disclosure are beneficial in healthcare settings, when temporary markings are needed, but supplies cannot be used from one patient to another because of worries related to contamination, helping to reduce hospital acquired infections.

In accordance with various aspects of the present disclosure, single-use wax marking tool may comprise a biodegradable plastic with a wax formulation that is safe for use on skin and hair. The marking tool prioritizes patient safety through infection and cross-contamination prevention.

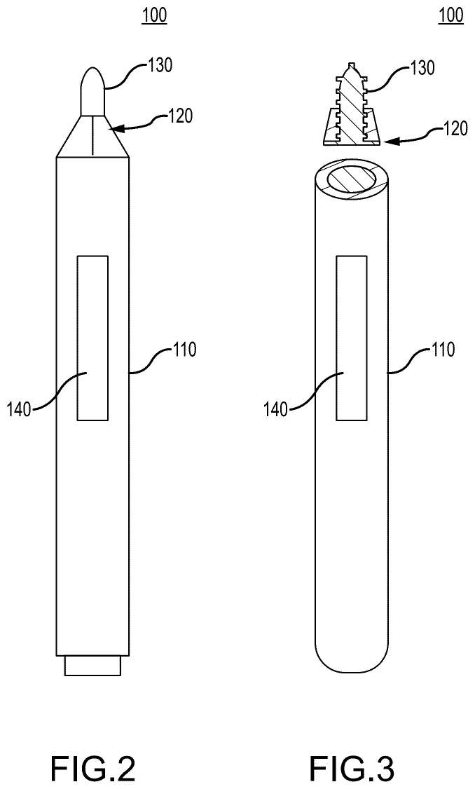

With reference to FIG. 2, an example of a single-use wax marking tool 100 is illustrated. For example, in accordance with various aspects of the present disclosure, a marking tool 100 has a small, hollow or mostly hollow body 110 marking tool with an end having an integrated hollow tapered tip 120 filled with a colored wax 130 inserted into and secured in the hollow tapered tip 120.

Alternatively, and with reference to FIG. 3, an example of a single-use wax marking tool 100 having a body 110 and a separate hollow tapered tip 120 is illustrated. In this example, in accordance with various aspects of the present disclosure, a marking tool 100 has a small, hollow or mostly hollow body 110 marking tool with a replaceable, attachable and detachable hollow tapered tip 120 filled with a colored wax 130 that is secured to the body 110.

In any of the various embodiments contemplated herein, the body 110 may alternatively be solid or mostly solid, rather than hollow or mostly hollow.

In accordance with various aspects of the present disclosure, the hollow tapered tip 120, whether integral or separate from the body 110, comprises a hollow portion to receive the wax. In various embodiments, the hollow tapered tip 120 may have structures that help secure the wax within the hollow tapered tip, though the wax may alternatively be press fit. For example, a projection within the hollow tapered tip 120 may help “grasp” and secure the wax within the hollow tapered tip 120. In other embodiments, other means for securing the wax within the hollow tapered tip 120 may be used and fall within the scope of the present disclosure, for example by press-fit, snap-fit, threads, glues or adhesives, suction and the like.

In accordance with various aspects of the present disclosure, the body 110 may be manufactured in any suitable manner from any suitable materials, such as various polymers. For example, the body 110 may be 3D printed from polylactic acid (PLA) which is beneficially biodegradable, though other suitably biodegradable materials and other methods of manufacture may be substituted and still fall within the scope of the present disclosure. However, in other embodiments, such as those where the hollow tapered tip 120 is separate, attachable and detachable from the body 110, the body 110 may comprise other materials, such as those more suitable for sterilization and disinfection (e.g., in an autoclave), including various metals such as stainless steel, as well as other materials resistant to high heat and disinfecting chemicals.

In various embodiments, the body 110 may have a printed label 140 designating various aspects or properties of the marking tool 100. The label 140 may or may not be biodegradable. Alternatively, the body 110 may have embossed or debossed labels 140 which further facilitate being entirely biodegradable body 110.

In accordance with various aspects of the present disclosure, the ingredients and formulations for the wax 130 are approved for use on skin and hair, are water and sweat resistant, and have a creamy consistency which helps minimize the amount of pressure placed on delicate skin when marking a patient, and easily wiped off the patient.

In accordance with the present disclosure, the body 110 may have a length of approximately 50 mm, though the particular length may vary based on the particular needs of the user, such as a smaller “pincher” option sized at approximately 25 mm. The body 110 may be generally cylindrical, the other shapes such as hexagonal, octagonal and the like may also be used, for example, to improve drip and comfort. Likewise, other uniform and non-uniform ergonomic grip shapes may be used in connection with the body 110. In cylindrical embodiments, the body is generally about 5 mm to 10 mm in diameter, though other diameters may be used based on the particular needs of the user.

The body 110 may be offered in red and blue colors as they are two commonly used colors in labs such as EEG labs though the colors may be expanded, and custom orders of varying sizes and colors may be contemplated.

In accordance with various aspects of the present disclosure, the marking tool 100 may be provided in the form of a kit having the body 110 and multiple, replaceable hollow tapered tips 120, for example as illustrated in FIG. 3. Similarly, a tip package having multiple hollow tapered tips 120 may be provided separate from the body or kit as refills for the marking tool 100 and body 110. The tip package 120 may have multiple hollow tapered tips that are the same color or different colors.

Finally, it will be apparent to those skilled in the art that various modifications and variations can be made in the present disclosure without departing from the spirit or scope of the disclosure. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, deposition materials, elements, components, shape, size and arrangement of parts including combinations within the principles of the invention, to the full extent indicated by the broad, general meaning of the terms in which the appended claims are expressed. To the extent that these various modifications do not depart from the spirit and scope of the appended claims, they are intended to be encompassed therein.

I claim:

1. A single-use wax marking tool comprising a hollow body having a hollow tapered tip having a colored wax therein to form the single-use wax marking tool.

2. The single-use wax marking tool of claim 1, wherein the colored wax is secured within the hollow tapered tip by press fit.

3. The single-use wax marking tool of claim 1, wherein the hollow tapered tip further comprises a projection therein to secure the colored wax within the hollow tapered tip.

4. The single-use wax marking tool of claim 1, wherein the hollow body has a length of about 25 mm to about 50 mm.

5. The single-use wax marking tool of claim 4, wherein the body is cylindrical.

6. The single-use wax marking tool of claim 5, wherein the body has a diameter of about 5 mm to about 10 mm.

7. The single-use wax marking tool of claim 1, wherein the wax is at least one of blue or red.

8. The single-use wax marking tool of claim 1, wherein the hollow body further comprises a label.

9. The single-use wax marking tool of claim 8, wherein the label is at least one of a printed label, an embossed label, or a debossed label.

10. The single-use wax marking tool of claim 9, wherein the label is biodegradable.

11. The single-use wax marking tool of claim 1, wherein the hollow body and the hollow tapered tip are separate attachable components.

12. The single-use wax marking tool of claim 11, further comprising a kit having the hollow body and multiple hollow tapered tips having the colored wax.

13. The single-use wax marking tool of claim 12, wherein the hollow body comprises stainless steel.

14. The single-use wax marking tool of claim 12, wherein at least one of the hollow body, the hollow tapered tips, and the wax is biodegradable.

15. The single-use wax marking tool of claim 11, further comprising a tip package having multiple hollow tapered tips.

16. The single-use wax marking tool of claim 15, wherein the multiple hollow tapered tips are the same color.

17. The single-use wax marking tool of claim 15, wherein the multiple hollow tapered tips are different colors.
